(19) United States
(12) Patent Application Publication
(10) Pub. No.: US 2025/0381394 A1
(43) Pub. Date: Dec. 18, 2025

(54) System and Method for Electro-Manual Therapy

(71) Applicant: Eric Manalansan, Frisco, TX (US)

(72) Inventor: Eric Manalansan, Frisco, TX (US)

(21) Appl. No.: 19/237,497

(22) Filed: Jun. 13, 2025

Related U.S. Application Data
(60) Provisional application No. 63/660,370, filed on Jun. 14, 2024.

(51) Int. Cl. A61N 1/32; A61H 7/00; A61N 1/08

(52) U.S. Cl. CPC A61N 1/322; A61H 7/003; A61N 1/08; A61H 2201/10; A61H 2201/1253

(57) ABSTRACT

A system and method for performing electro-manual therapy is disclosed. In one embodiment, an electrode is attached to a practitioner, allowing the practitioner to deliver current through his hand or a bare-handed grip on a conductive tool. In another embodiment, an electrode is attached directly to the conductive tool, which can be held by a practitioner with a gloved hand. For all embodiments, a signal generator can automatically detect when contact with the patient is broken and ramps down the current to ensure patient comfort. This allows for the safe, effective, and simultaneous application of manual therapy and electrical stimulation.

### FIELD OF INVENTION

The present disclosure relates to the field of physical therapy, and more specifically to systems and methods for combining electrotherapy with manual therapy.

### BACKGROUND OF THE INVENTION

Conventionally, manual therapy techniques and electrotherapy have been applied as separate and distinct procedures. A practitioner might use a mechanical tool for instrument-assisted soft tissue mobilization (IASTM) and then, in a separate step, apply adhesive electrodes to the patient for electrical stimulation. This passive approach fails to exploit the synergistic benefits that can arise when electrical stimulation is combined with active, hands-on manual therapy.

Methods that attempt to integrate these therapies by passing an electrical current through the practitioner are known. In such systems, the practitioner becomes part of the electrical circuit, delivering current through their hands or a conductive glove. However, a drawback of these systems is the management of the electrical current during treatment. When the practitioner breaks and re-establishes physical contact with the patient, the abrupt start or stop of the current flow can cause discomfort or a shocking sensation for the patient.

Therefore, a need exists for an integrated electro-manual therapy system that improves upon these previous methods by enhancing patient safety and comfort, while simplifying the procedure for the practitioner.

### SUMMARY OF THE INVENTION

The present invention seeks to solve the problem that existing electro-manual therapy methods can cause patient discomfort. This problem is solved by an electrotherapy system that automatically senses when practitioner-guided contact with the patient is broken and instantly ramps the current down, ensuring a safe and seamless treatment.

Accordingly, in a first embodiment, a method for performing electro-manual therapy involves attaching an electrode to the practitioner, who then becomes part of the electrical pathway. The practitioner contacts a patient using a conductive interface, such as the practitioner's bare hand or a conductive tool held in their bare hand. The method is characterized by the system detecting when this contact is broken and, in response, automatically ramping down the electrical current.

In a second embodiment, a method for performing electro-manual therapy involves attaching an electrode directly to a conductive manual therapy tool. A practitioner, who is not part of the electrical pathway, holds and manipulates the tool to treat the patient. The practitioner may hold the tool with a gloved hand for comfort and insulation. In this embodiment as well, the system can detect when the tool breaks contact with the patient and automatically ramp down the current.

A system for performing this therapy is also disclosed. The system comprises a signal generator and can be configured for either embodiment: with an electrode attachable to the practitioner for use with a bare hand or a bare-handed grip on a conductive tool, or with an electrode attachable directly to a conductive IASTM tool for use by a practitioner who may be wearing a glove.

### BRIEF DESCRIPTION OF THE DRAWINGS

FIG. 1 is a diagram of the electro-manual therapy system 10 in an embodiment where the practitioner is part of the conductive pathway, and the practitioner's hand 25 serves as the conductive interface.

FIG. 2 is a diagram of the electro-manual therapy system 10 in an embodiment where the practitioner is part of the conductive pathway, and a conductive IASTM tool 28 held by the practitioner's bare hand serves as the conductive interface.

FIG. 3 is a perspective view of an exemplary conductive IASTM tool 28, which may be similar in form to a Gua Sha knife, illustrating its handle portion and tapered treatment edge.

FIG. 4 is a logic block diagram illustrating the control process for the automatic current ramping feature, including a contact detection module 37.

FIGS. 5A and 5B are diagrams of the electro-manual therapy system 10 in an alternative embodiment where an electrode 46 is connected directly to the conductive IASTM tool 28, and the practitioner 16 holds the tool with a bare hand and a gloved hand respectively.

### DETAILED DESCRIPTION OF THE PREFERRED EMBODIMENT

The present invention provides a system and method for safely and effectively integrating electrotherapy with practitioner-guided manual therapy. The system is designed to allow for the delivery of a therapeutic electrical current to a patient through either the practitioner's own hand or through a specialized tool, and includes a safety feature to automatically manage the current upon making and breaking contact with the patient.

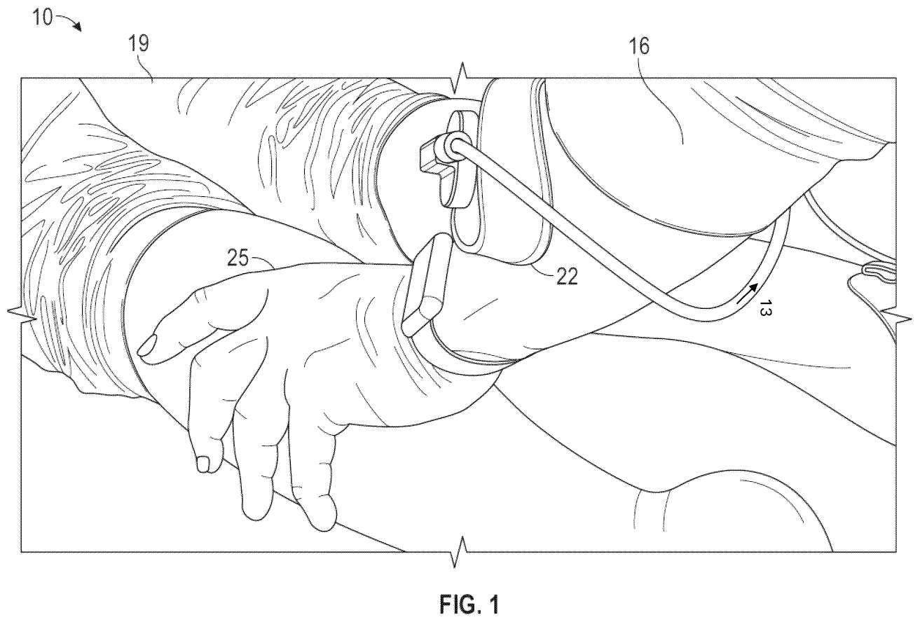

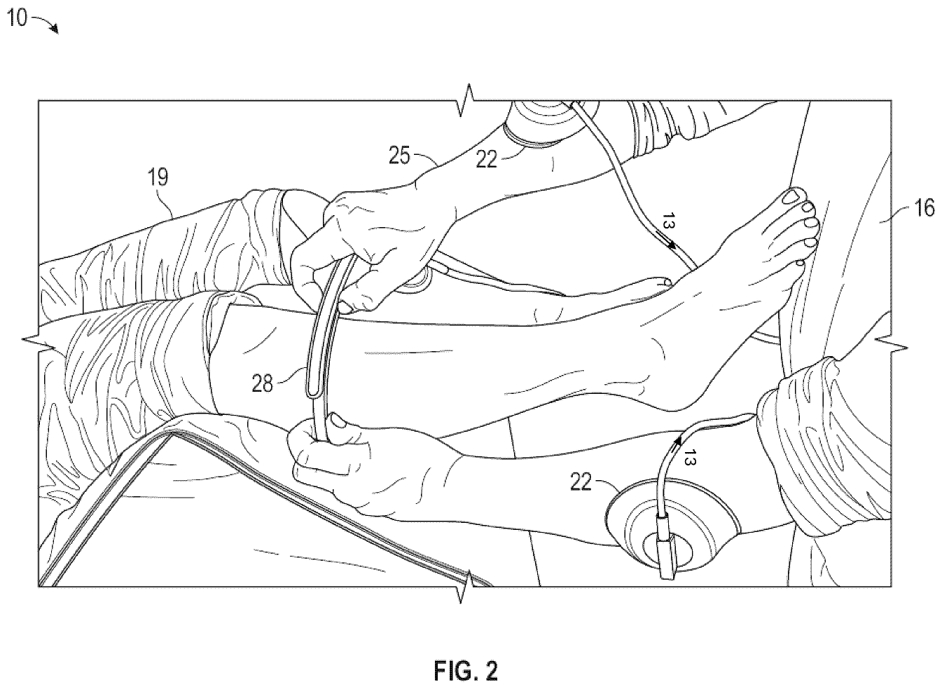

In a first general embodiment, illustrated in FIG. 1 and FIG. 2, the practitioner 16 acts as a conduit for the therapeutic electrical current. Referring to FIG. 1, the system 10 includes a signal generator electrically connected to at least one electrode 22. This practitioner-worn electrode 22 is configured to be attached to a body part of a practitioner 16, such as a forearm, making the practitioner 16 part of the electrical pathway. In use, the practitioner 16 contacts a patient 19 with a conductive interface. In the embodiment of FIG. 1, the conductive interface is the practitioner's own hand 25. This contact establishes a conductive pathway for current to flow from the signal generator, through the electrode 22, through the practitioner 16, and through the hand 25 to the patient 19.

Referring to FIG. 1, a general embodiment of the electro-manual therapy system 10 is shown. The system 10 includes a signal generator 13, which produces a “therapeutic electrical current.” A therapeutic electrical current, as used herein, is an electrical current specifically configured to elicit desired physiological responses in a patient 19 for therapeutic purposes, such as, but not limited to, muscle re-education, pain modulation (e.g., reducing acute or chronic pain), reduction of inflammation, and improvement of muscle function.

The characteristics of a therapeutic electrical current are chosen to be both safe and effective for these purposes. For instance, a suitable current may be a pulsed alternating current, where the current is delivered in a series of short bursts. To optimize therapeutic outcomes and ensure patient comfort, the parameters of this current are often modulated. This can include frequency modulation, where the number of pulses or cycles per second is varied during the treatment. This method is commonly used in electrotherapy to prevent the patient's nervous system from adapting or accommodating to the stimulation, thereby maintaining the treatment's effectiveness. Other suitable modulated waveforms known in the art may also be used.

The parameters of this therapeutic electrical current, including its waveform (preferably sinusoidal), frequency (preferably within a range of 0.1 Hz to 33,000 Hz), intensity amplitude or voltage (preferably within a range of 1 μA to 100 mA and held at a constant output), and duration of application (typically from 1 minute to 30 minutes), are typically adjustable by the practitioner 16 via the signal generator to tailor the treatment to the specific needs and condition of the patient 19 and the intended therapeutic goals. It is preferred that the current be adjustable at the signal generator for both amplitude and frequency. Constant voltage output and a balanced sinusoidal waveform are also preferred.

As used herein, the term signal generator refers to an electrotherapy device or unit capable of producing and outputting such a therapeutic electrical current with specific, controllable parameters. The signal generator may incorporate control circuitry, user interfaces (such as dials, buttons, or a digital display as depicted in the inventor's provisional disclosures), and power supply components to generate the desired electrical output. In the context of the present invention, the signal generator is further characterized by its connection to the practitioner-worn electrode 22 and its role in the automatic current ramping feature.

The signal generator is electrically connected to at least one electrode 22. As used herein, at least one electrode 22 refers to an electrically conductive element or pad designed to be securely attached to a body part of a practitioner 16, such as a forearm, and to make electrical contact between the signal generator and the practitioner 16, thereby allowing the practitioner 16 to become part of the electrical pathway. This electrode 22 is configured to be attached to a body part of a practitioner 16, such as a forearm. In use, the practitioner 16 contacts a treatment area on a patient 19 with a conductive interface. As used herein, conductive interface refers to the electrically conductive medium or object that the practitioner 16 uses to make direct physical contact with the patient 19 and through which the therapeutic electrical current is transferred from the practitioner 16 to the patient 19.

In the embodiment shown in FIG. 1, the conductive interface is the practitioner's own hand 25. This contact establishes a conductive pathway enabling current to flow from the signal generator, through the electrode 22 attached to the practitioner 16, through the practitioner 16, through the conductive interface (e.g., hand 25) contacting the patient 19, and into the patient 19. For the therapeutic electrical current to be effective and for the system 10 to operate as intended, a complete electrical circuit is typically formed, often via one or more separate return electrodes (not shown) placed on the patient 19 and also connected to the signal generator, thereby providing a path for the current back to the signal generator.

Referring to FIG. 2, in a variation of this first embodiment, the conductive interface is a handheld, conductive instrument-assisted soft tissue mobilization (IASTM) tool 28. As used herein, an instrument-assisted soft tissue mobilization (IASTM) tool 28 refers generally to a class of manual therapy instruments, which can include tools similar in form and mechanical function to those used in traditional practices like Gua Sha (e.g., a Gua Sha knife or scraper) or in modern IASTM techniques. These tools are designed to be held by a practitioner 16 and applied to a patient's 19 skin to exert specific mechanical forces (such as compression, stroking, or shear) on underlying soft tissues, including muscles, fascia, and tendons. Such tools are typically used to detect and treat fascial restrictions, scar tissue, and areas of chronic inflammation. In the context of the present invention, the IASTM tool 28 is further characterized as being handheld for ease of manipulation by the practitioner 16, and, critically, as being conductive. This means the body of the IASTM tool 28 itself is made from an electrically conductive material, or incorporates sufficient conductive elements, to allow it to serve as the point of current transfer to the patient 19. The practitioner 16 holds the conductive IASTM tool 28 in a bare hand, and the electrical current passes from the signal generator, through the practitioner 16 (via the attached electrode 22), and into the tool 28, which then contacts the patient 19. This allows the practitioner 16 to simultaneously deliver the mechanical shear forces characteristic of IASTM and the therapeutic benefits of electrical stimulation through a single instrument.

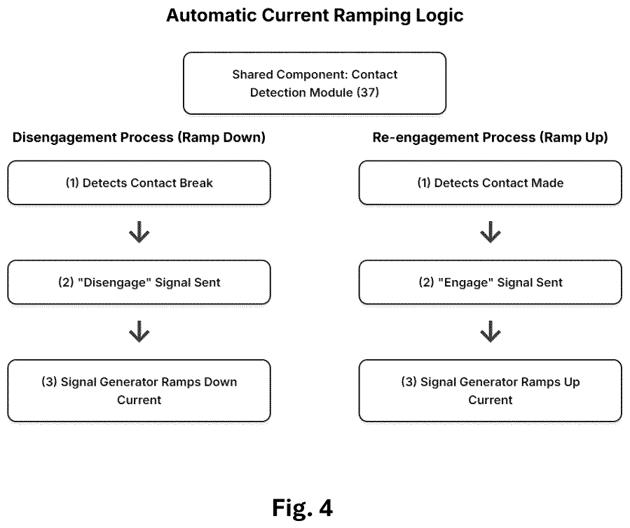

A primary inventive feature applicable to all embodiments is the logic for managing the electrical current. The signal generator is configured to detect a disengagement of the conductive interface (e.g., hand 25 or tool 28) from the patient 19. As shown in the logic diagram of FIG. 4, this detection can be performed by a contact detection module 37 that monitors the electrical properties of the pathway, such as impedance. When the conductive pathway is interrupted, the signal generator automatically ramps down the electrical current. The system 10 may also be configured to detect re-engagement and automatically ramp the current back up.

The detection of disengagement (i.e., an interruption of the conductive pathway to the patient 19) by the contact detection module 37 or equivalent logic within the signal generator can be achieved by various means known in the art of electronics. For example, the signal generator may include circuitry to monitor the electrical impedance across its output terminals. A sudden and significant increase in impedance above a predetermined threshold would indicate that the conductive interface is no longer in sufficient contact with the patient 19, thereby signaling a disengagement. Alternatively, a low-level continuity signal could be monitored through the established pathway; interruption of this signal would signify a disengagement. Once disengagement is detected, the control circuitry within the signal generator, which may include a microcontroller, is programmed to initiate a gradual reduction of the output current amplitude over a short period, rather than an instantaneous cut-off. This “ramping down” can be achieved by, for example, digitally controlling an attenuator, adjusting the gain of an output amplifier, or modulating the power supply to the current-generating components. Similarly, upon detecting re-engagement (e.g., by observing a return of impedance to within an expected range or re-establishment of the continuity signal), the control circuitry can be programmed to gradually increase the current amplitude back to the set therapeutic level. Such control mechanisms are within the purview of one skilled in the art of electronic circuit design and software programming for medical or therapeutic devices.

In one possible implementation, the contact detection module 37 can be configured to detect disengagement by leveraging principles analogous to Bioelectrical Impedance Analysis (BIA). While conventional BIA is used to estimate tissue composition, an embodiment of the present system could adapt this principle for a novel purpose. Rather than interpreting the absolute impedance value for physiological analysis, the system 10 could establish a stable baseline impedance when the conductive interface (25, 28) is in firm contact with the patient 19. A disengagement could then be registered by the contact detection module 37 upon detecting a sudden and substantial increase in impedance above a predetermined threshold. This change from a relatively low-impedance state to a very high-impedance state (an air gap) would provide a reliable trigger for the control circuitry to automatically ramp down the therapeutic current.

As used herein, the term “ramping down” refers to the process by which the signal generator automatically initiates a gradual reduction of the electrical current's amplitude over a short period. This process is a controlled decrease in intensity, as opposed to an instantaneous cut-off of the current. The primary purpose of ramping down, which is triggered upon detecting a disengagement between the conductive interface and the patient, is to prevent patient discomfort or a shocking sensation that can result from an abrupt interruption of the current flow.

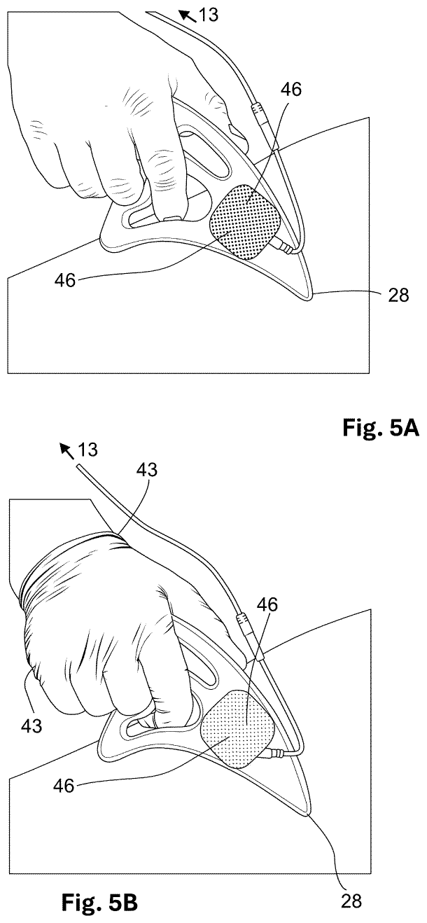

In a second general embodiment, illustrated in FIG. 5, the practitioner 16 is electrically isolated from the therapeutic current. In this configuration, the system 10 includes an electrode 46 that is configured to connect directly from the signal generator to the conductive IASTM tool 28. The practitioner 16 holds and manipulates the IASTM tool 28 but is not part of the primary conductive pathway. Because the practitioner 16 is not intended to conduct the current, they may hold the IASTM tool 28 with a gloved hand, using a standard insulating glove 43 for comfort and to ensure they are electrically isolated. In this embodiment, the conductive IASTM tool 28 itself is the active electrode that delivers current to the patient 19.

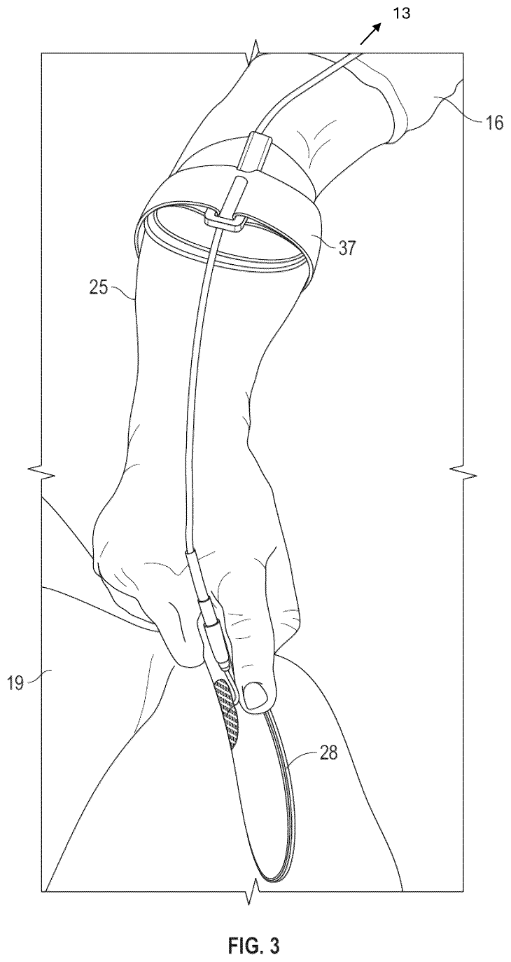

As shown in FIG. 3, the conductive IASTM tool 28 may be an ergonomically shaped instrument having a handle portion for gripping and a tapered treatment edge for application to the patient's 19 tissue. The form of such a tool 28 may resemble that of a traditional Gua Sha knife, adapted or constructed from a conductive material. While the overall shape may be similar to known IASTM or Gua Sha tools, the tool 28 of the present system 10 is made of a conductive material (e.g., stainless steel, conductive polymer, or other suitable conductive metals or materials) to allow for the passage of the therapeutic electrical current from the tool 28 to the patient 19 upon contact.

In some embodiments, as shown in FIG. 5, the practitioner 16 may hold the IASTM tool 28 with a gloved hand. The glove 43 worn by the practitioner 16 in this embodiment is typically a standard examination glove or a glove 43 made of an electrically insulating material.

The purpose of the glove 43 is to provide a barrier that prevents or minimizes the practitioner's 16 perception of the electrical current from the energized conductive IASTM tool 28. This may be preferable for practitioners 16 who are sensitive to the sensation of the current or wish to avoid perceiving it for other reasons. Importantly, in this gloved hand embodiment, the electrical current is still delivered to the patient 19 via the conductive IASTM tool 28 itself, which remains the conductive interface making direct contact with the patient 19. The glove 43 merely interfaces between the practitioner's 16 skin and the handle of the conductive IASTM tool 28.

While the invention has been described in connection with what are presently considered to be the most practical and preferred embodiments, it is to be understood that the invention is not to be limited to the disclosed embodiments. On the contrary, it is intended to cover various modifications and equivalent arrangements included within the spirit and scope of the appended claims. The scope of the claims is to be accorded the broadest interpretation so as to encompass all such modifications and equivalent structures and functions.

What is claimed is:

1. A method for performing electro-manual therapy, the method comprising:
attaching at least one electrode to a body part of a practitioner, wherein the at least one electrode is electrically connected to a signal generator;
the practitioner contacting a patient with a conductive interface, thereby establishing a conductive pathway for an electrical current to flow from the signal generator, through the at least one electrode, through the practitioner, and through the conductive interface to the patient;
detecting a disengagement of the conductive interface from the patient, wherein the disengagement interrupts said conductive pathway; and
in response to detecting the disengagement, automatically ramping down the electrical current from the signal generator.

2. The method of claim 1, further comprising detecting a re-engagement of the conductive interface with the patient and, in response, automatically ramping up the electrical current.

3. The method of claim 1, wherein the conductive interface is the practitioner's hand.

4. The method of claim 1, wherein the conductive interface is a handheld, conductive instrument-assisted soft tissue mobilization (IASTM) tool held in a bare hand of the practitioner.

5. A method for performing electro-manual therapy, the method comprising:
attaching at least one electrode directly to a handheld, conductive instrument-assisted soft tissue mobilization (IASTM) tool, wherein the at least one electrode is electrically connected to a signal generator;
a practitioner holding the IASTM tool and contacting a patient with the IASTM tool, thereby establishing a conductive pathway for an electrical current to flow from the signal generator, through the at least one electrode, and through the IASTM tool to the patient;
detecting a disengagement of the IASTM tool from the patient; and
in response to detecting the disengagement, automatically ramping down the electrical current from the signal generator.

6. The method of claim 5, wherein the practitioner holds the IASTM tool with a gloved hand.

7. A system for performing electro-manual therapy, the system comprising:
a signal generator;
at least one electrode configured for attachment to a body part of a practitioner and electrically connected to the signal generator;
wherein the system is configured to establish a conductive pathway for an electrical current to flow from the signal generator, through said electrode, through the practitioner, and through a conductive interface to a patient; and
wherein the signal generator is further configured to detect a disengagement of the conductive interface from the patient and automatically ramp down the electrical current in response.

8. The system of claim 7, wherein the conductive interface is the practitioner's hand.

9. The system of claim 7, wherein the conductive interface is a handheld, conductive instrument-assisted soft tissue mobilization (IASTM) tool configured to be held in a bare hand of the practitioner.

10. The system of claim 9, wherein the IASTM tool comprises a body having a handle portion and a tapered treatment edge.

11. A system for performing electro-manual therapy, the system comprising:
a signal generator;
a handheld, conductive instrument-assisted soft tissue mobilization (IASTM) tool;
at least one electrode configured to electrically connect the signal generator directly to the conductive IASTM tool;
wherein the system is configured to establish a conductive pathway through the conductive IASTM tool to a patient; and
wherein the signal generator is further configured to detect a disengagement of the IASTM tool from the patient and automatically ramp down the electrical current in response.

12. The system of claim 11, further comprising a glove configured to be worn by a practitioner while holding the IASTM tool.
